(19) United States
(12) Patent Application Publication
(10) Pub. No.: US 2025/0312419 A1
(43) Pub. Date: Oct. 9, 2025

(54) PHARMACEUTICAL COMPOSITIONS AND METHODS

(71) Applicant: Mannkind Corporation, Danbury, CT (US)

(72) Inventors: Ryan D. Soracco, Seymour, CT (US); John J. Freeman, Jr., New Fairfield, CT (US); Grayson W. Stowell, Holly Springs, NC (US); Logan D. Pennarola, Danbury, CT (US); Marshall L. Grant, Newtown, CT (US)

(21) Appl. No.: 18/728,814

(22) PCT Filed: Jan. 11, 2023
(86) PCT No.: PCT/US2023/010549

§ 371 Date: Jul. 12, 2024

Related U.S. Application Data
(60) Provisional application No. 63/266,718, filed on Jan. 12, 2022.

(51) Int. Cl. A61K 38/22; A61K 9/00; A61K 9/16; A61K 47/18; A61K 47/22

(52) U.S. Cl. CPC A61K 38/2278; A61K 9/0075; A61K 9/1611; A61K 9/1617; A61K 9/1623; A61K 47/183; A61K 47/22

(57) ABSTRACT

Disclosed herein are stable solution, suspension and dry powder compositions, and methods for delivering stabilized biodegradable substances, including peptides and proteins, small molecules, and methods for delivering the dry powders in the treatment of lung disease. In particular, the compositions comprise vasoactive intestinal peptide for pulmonary inhalation to treat respiratory disorders and/or diseases in the lung, including, inflammation, acute and chronic lung injury and pulmonary edema.

### TECHNICAL FIELD

Disclosed herein are stable pharmaceutical compositions and methods for delivering biodegradable substances, including peptides and proteins, and methods for delivering the dry powders in the treatment of lung disease. In particular, solutions, suspensions and dry powders are intended for pulmonary inhalation to treat respiratory disorders and/or diseases in the lung, including, inflammation, edema, acute and chronic lung injury.

### BACKGROUND

Delivery of unstable drugs to treat disease has been a major problem for many years. Many compounds are ineffective or exhibit low or variable potency when they are administered orally. With oral administration of unstable compounds, there can be diminished absorption of the compounds under the conditions encountered in the gastro-intestinal tract and thus diminished activity prior to reaching their targeted location. While it is preferable in many cases to administer drugs orally especially in terms of ease of administration, patient compliance, and decreased cost, alternative methods are needed to decrease drug content used per tablet or capsule, since increased drug content may lead to adverse effects. For biologic products, in particular, peptides and proteins, the acidic environment in the stomach is detrimental to maintain their function, as most proteins degrade readily.

Drug formulations comprising unstable drugs, including isolated biological substances, including, proteins and peptides are most commonly formulated as injectable products. In the injectable formulations, however, these unstable compounds can readily undergo denaturation and can completely lose functional activity, by entering the venous circulation and passing through the liver where they can be metabolized. Some biologic products, for example, lose functional activity by taking them out of −20° C. storage and placed at room temperature for a short period of time. Other isolated proteins and peptides undergo significant degradation when stored at 4° C., without the addition of protease inhibitors, due to oxidation or ubiquitously occurring proteases. Most mammalian proteins and peptides degrade at a temperature greater than 43° C. It has been well established that at 55° C., most proteins undergo complete denaturation in about 1-2 hours. In some cases, complete denaturation and destabilization of an isolated protein also occurs at room temperature.

Currently, formulations containing unstable drug products as active agents for treating local and systemic disease for delivery to the lungs are available primarily through injectable compositions, and solutions and suspensions for nebulization. The preparation of injectable formulations and special storage needed create challenges, which prohibit their use, in particularly, in subtropical and tropical climates where there is a great need, and refrigeration and sterilization are not always readily available. In particular, alternative methods and formulations are needed to stabilize biologically-derived products for the treatment of subjects, including, during a pandemic occurrence of disease such as corona virus disease, including, Covid-19.

Dry powder composition for pulmonary inhalation and systemic delivery of peptides, for example, insulin (AFREZZA®), requires initially cold temperature storage before use. While AFREZZA can be stored at ambient temperature for short periods of time, e.g., weeks to months, there is still a need to improve stability of peptide in dry powder products. Similarly, there is a need to improve the stability of dry powder compositions intended for lung delivery, especially those comprising a biologic molecule to further prolonged their shelf-life, facilitate their storage and delivery prior to patient use, particularly, if refrigeration is not available.

Therefore, there is room for improvement in the development of pharmaceutical formulations comprising biologic molecules in particular for pulmonary delivery in the treatment of disease.

### SUMMARY

The present disclosure provides a method for treating an acute, or a chronic lung disease or disorder, including, inflammation, pulmonary fibrosis, viral infections, including, Covid-19 disease. The method comprises administering to a subject a pharmaceutical composition, including, suspensions and dry powder composition for inhalation comprising an unstable active agent, which composition has improved stability of the active agent, including, in solution or suspension, at room temperature, or higher temperatures for prolonged periods of time, without substantially losing biological activity by degradation of the active agent.

In one embodiment, an inhalable pharmaceutical formulation is provided comprising a dry powder for inhalation comprising, an unstable, rapidly degrading, small molecule or large molecule, including, a biologic molecule, wherein the biologic molecule, including, a peptide, a protein, in particular of synthetic origin and the biologic molecule is easily destabilized in aqueous solution or suspension. In an exemplary embodiment, the pharmaceutical formulation is manufactured for inhalation to local lung tissue. The formulation is provided for delivery to the lungs using a dry powder inhalation system comprising a multiple use inhaler that can be used with a replaceable unit dose cartridge or capsule. Alternatively, a single use inhaler with an integrally built-in container can be provided containing the formulation for single use, or a multidose inhaler can also be provided with a plurality of doses integrally configured with the inhaler for multiple uses.

In another embodiment, a stable inhalable pharmaceutical formulation is provided comprising, a dry powder comprising a protein or a peptide and one or more pharmaceutically acceptable carriers and/or excipients, which formulations are stable at room temperature, high temperatures and/or high humidity. In one embodiment, the pharmaceutical formulation is stable for a long period of time at temperatures, for example, temperatures greater than 4° C., greater than 10° C., greater than 20° C., or greater than 35° C. In one embodiment, the formulations are stable at relative humid environments such as environments having a relative humidity greater than 5%, greater than 10%, greater than 30%, greater than 50%, greater than 60%, greater than 70%.

In an exemplary embodiment, a pharmaceutical formulation for use in the method of treatment of lung disease is stabilized by one or more pharmaceutically acceptable carriers and/or excipients, or combinations thereof, and include, for example, buffers, salts, minerals, vitamins, antioxidants, polymers, sugars, including, mannitol, xylitol, diketopiperazines and/or salts thereof, and the like. In one embodiment, the dry powder compositions can, optionally, include, surfactants such as polysorbates, for example, polysorbate 80 and Tween.

In certain embodiments, the formulation comprises a dry powder comprising a therapeutic peptide for treating inflammatory disease such as pulmonary edema, including, vasoactive intestinal peptide (VIP), a derivative thereof, an analog thereof, a pharmaceutically acceptable salt thereof, or combinations thereof, including, aviptadil acetate, and one or more pharmaceutical excipients and/or carriers, including, peptide stabilizing agents. In an example embodiment, the pharmaceutically acceptable carrier and/or excipient can be formulated for oral inhalation, which can form particles suitable for inhalation, example, diketopiperazines, including, fumaryl diketopiperazine; wherein the particles form amorphous powders, a crystalline powders, or a crystalline composite powders.

In one embodiment, the pharmaceutically acceptable carrier or excipient comprises, one or more sugars, including, mannitol, xylitol, sorbitol, and trehalose; one or more amino acids, including, arginine, glycine, leucine, isoleucine, trileucine, cysteine, lysine, methionine and histidine; surfactants, including, polysorbate 80; cationic salts, including, monovalent, divalent and trivalent salts, including, sodium chloride, potassium chloride, magnesium chloride, and zinc chloride; buffers, including, citrates and tartrates, vitamins, including, vitamin A, vitamin C, vitamin E, or combination of one or more carriers and/or excipients and the like.

In a particular embodiment, the pharmaceutical dry powder composition comprises one or more pharmaceutical excipients and/or carriers, including antioxidants, for example, ascorbic acid or vitamin C, vitamin E, selenium, carotenoids, including, beta-carotene, lycopene, lutein, and zeaxanthin; glutathione, phenols, polyphenols, and flavonoids.

In a particular embodiment, a pharmaceutical dry powder composition comprises vasoactive intestinal peptide (VIP), a derivative thereof, an analog thereof, a pharmaceutically acceptable salt thereof, and/or combinations thereof, including, aviptadil; aviptadil acetate, a sugar, an amino acid and/or an antioxidant; wherein the sugar is mannitol, or trehalose; the amino acid is leucine, isoleucine, methionine, or combinations thereof. In certain embodiments herewith, the pharmaceutical dry powder composition further comprises an antioxidant selected from the group consisting of vitamin C, vitamin E, selenium, and glutathione.

In an exemplary embodiment, the pharmaceutical dry powder composition comprises vasoactive intestinal peptide (VIP), a derivative thereof, an analog thereof, a pharmaceutically acceptable salt thereof, and/or combinations thereof, including, aviptadil; aviptadil acetate, wherein the one or more pharmaceutically acceptable carrier and/or excipients, include, mannitol, methionine, leucine and ascorbic acid.

In other embodiments, the inhalable pharmaceutical dry powder can further comprise an inorganic salt or an organic salt or combinations thereof, including, sodium chloride, potassium chloride, magnesium chloride, zinc chloride, sodium citrate, sodium tartrate, or combinations thereof.

In one embodiment, an inhalable dry powder composition can comprise a buffer, and a monovalent or divalent cationic salt. In a particular embodiment, the formulation comprises a dry powder comprising VIP, a VIP derivative, a VIP analog, or a salt thereof; a buffer and/or a divalent cation or monovalent cation provided by a salt, including, zinc citrate, zinc acetate, disodium tartrate, mono-sodium tartrate, sodium citrate, disodium citrate, trisodium citrate, zinc chloride, calcium chloride, magnesium chloride, sodium hydroxide, and the like. In an embodiment, the monovalent cation in the compositions, include, sodium, potassium and lithium. In an alternate embodiment, the formulation can be provided with citric acid as the excipient.

In a specific embodiment, a dry powder composition is provided comprising and active agent such as vasoactive intestinal peptide in an amount less than 40 wt %, less than 30 wt %, less than 20 wt wt %, or less than 10 wt %, and a sugar in an amount less than 90 wt %, less than 85 wt %, or less than 50 wt % in the composition. In a particular embodiment, the amino acid component in the composition is in an amount up to about 20 wt %, up to about 15 wt %, or up to about 10 wt % in the composition. In an embodiment, the amount of antioxidant in the composition can be up to about 10%, up to 5 wt %, or up to 3% in the composition.

In a specific embodiment, an inhalable dry powder composition for the treatment of lung disease, including, an acute or chronic infection of the lungs comprises, vasoactive intestinal peptide in an amount of up to 50 wt %; wherein the pharmaceutically acceptable excipient or carriers are mannitol, leucine and ascorbic acid.

A method of making a dry powder formulation comprising mixing or homogenizing a solution comprising a peptide or protein or analog thereof, wherein the solution further comprises a sugar, including, mannitol, one or more amino acids, including, leucine or isoleucine, and an antioxidant, including, ascorbic acid or glutathione and spray drying the solution to form a dry powder.

A method for treating lung disease, comprising administering to a subject in need of treatment a dose of a dry powder composition by inhalation one or more times a day, using a dry powder inhaler, wherein the composition comprises vasoactive intestinal peptide, an analog thereof or derivative thereof; an amino acid, a sugar and an antioxidant. In one embodiment, the treatment comprises administrating one or more doses of the dry powder composition per session. In one embodiment herewith, a dose of the composition comprises up to about 500 μg, 300 μg, 200 μg, or 100 μg of vasoactive intestinal peptide per session.

In an exemplary embodiment, a method for treating lung disease comprises administering to a subject in need of treatment, including pulmonary edema, an inhalable pharmaceutical formulation comprising mannitol, leucine, methionine and vasoactive intestinal peptide, an analog thereof, or a derivative thereof in a dose of up to 200 μg per session at least once a day. In an embodiment, the pharmaceutical formulation can be a solution, suspension, or a dry powder for inhalation and can be administered by nebulization, metered dose inhaler or a dry powder inhaler.

In other embodiments described herewith, there are disclosed methods for making stable compositions comprising vasoactive intestinal peptide, derivatives thereof, analogs thereof, or combinations thereof, and other rapidly degraded bioactive agents, and method for using the compositions in the treatment of disease and/or disorders of the lungs or systemic origin. In an exemplary embodiment, an inhalation system is provided which includes a high resistance inhaler for single dose usage for the treatment of lung disease comprising the compositions.

In another embodiment, the method of treatment comprises administering to a subject in need of treatment, an inhalable pharmaceutical formulation comprising mannitol, leucine, methionine and vasoactive intestinal peptide, an analog thereof, or a derivative thereof in a dose of up to 200 μg per session at least once a day. In an embodiment, the pharmaceutical formulation can be a solution, suspension, or a dry powder for inhalation and can be administered by nebulization, metered dose inhaler or a dry powder inhaler. In this and other embodiments, the disease to be treated is, for example, including, pulmonary edema, Crohn's disease, heart failure, neurodegenerative disease and the like.

### BRIEF DESCRIPTION OF THE DRAWINGS

The following drawings form part of the present specification and are included to further demonstrate certain aspects of the examples disclosed herein. The disclosure may be better understood by reference to one or more of these drawings in combination with the detailed description of specific embodiments presented herein.

FIGS. 1A, 1B and 1C are scanning electron micrographs (SEM) of dry powders made comprising crystalline particles of fumaryl diketopiperazine and vasoactive intestinal peptide (VIP), wherein the dry powders contain differing amounts of the VIP peptide.

FIG. 2 illustrates a graphic representation of data from experiments for the standard curve of VIP in the dry powders.

FIG. 3 depicts HPLC chromatograms of the VIP peptide used in the experiments.

FIG. 4 illustrates HPLC chromatograms of the contents of the dry powders comprising crystalline FDKP and VIP from samples made by the methods described in Example 1 below.

FIG. 5 depicts a graphic illustration of formulation experiments conducted to test various types of solutions or suspensions as described in Example 2 comprising vasoactive intestinal peptide (VIP); showing the stability of VIP in the compositions with various methionine contents versus control samples.

FIG. 6 depicts a graphic illustration of formulation experiments conducted to test various types of solutions or suspensions (see Table 2 below) comprising vasoactive intestinal peptide (VIP) showing the stability of VIP in the compositions with various pharmaceutically acceptable excipients.

### DETAILED DESCRIPTION

Disclosed herein are pharmaceutical dry powders for inhalation comprising a vasoactive intestinal peptide, and methods for making the powder composition for the treatment of lung disease. Embodiments disclosed herein show that a rapidly biodegradable active agent including, vasoactive intestinal peptide can be formulated to be stable in a composition for treating lung disease, including, pulmonary edema.

Method for making dry powder formulations comprising stabilized vasoactive intestinal peptide is provided, which facilitates the use of VIP in treating disease, and in particular lung disease. A method of making stabilized VIP and the dry powder compositions therefrom are also provided.

As used herein, the term “microparticle” refers to a particle with a diameter of about 0.5 to about 1000 μm, irrespective of the precise exterior or interior structure. Microparticles having a diameter of between about 0.5 and about 10 microns can reach the lungs, successfully passing most of the natural barriers. A diameter of less than about 10 microns is required to navigate the turn of the throat and a diameter of about 0.5 microns or greater is required to avoid being exhaled. To reach the deep lung (or alveolar region) where most efficient absorption is believed to occur, it is preferred to maximize the proportion of particles contained in the “respirable fraction” (RF), generally accepted to be those particles with an aerodynamic diameter of about 0.5 to about 6 microns, though some references use somewhat different ranges, as measured using standard techniques, for example, with an Anderson Cascade Impactor. Other impactors can be used to measure aerodynamic particle size such as the NEXT GENERATION IMPACTOR™ (NGIT, MSP Corporation), for which the respirable fraction is defined by similar aerodynamic size, for example<6.4 μm. In some embodiments, a laser diffraction apparatus is used to determine particle size, for example, the laser diffraction apparatus disclosed in U.S. patent application Ser. No. 12/727,179, filed on Mar. 18, 2010, which is incorporated herein in its entirety for its relevant teachings, wherein the volumetric median geometric diameter (VMGD) of the particles is measured to assess performance of the inhalation system. For example, in various embodiments cartridge emptying of 80%, 85%, or 90% and a VMGD of the emitted particles of: 512.5 μm; 5 7.0 μm, or: 5 4.8 μm can indicate progressively better aerodynamic performance.

As used herein, the term “about” is used to indicate that a value includes the standard deviation of the measurement for the device or method being employed to determine the value.

Respirable fraction on fill (RF/fill) represents the % of powder in a dose that is emitted from an inhaler upon discharge of the powder content filled for use as the dose, and that is suitable for respiration, i.e., the percent of particles from the filled dose that are emitted with sizes suitable for pulmonary delivery, which is a measure of microparticle aerodynamic performance. As described herein, a RF/fill value of 40% or greater than 40% reflects acceptable aerodynamic performance characteristics. In certain embodiments disclosed herein, the respirable fraction on fill can be greater than 50%. In an exemplary embodiment, a respirable fraction on fill can be up to about 80%, wherein about 80% of the fill is emitted with particle sizes<5.8 μm as measured using standard techniques.

As used herein, the term “dry powder” refers to a fine particulate composition that is not suspended or dissolved in a propellant, or other liquid. It is not meant to necessarily imply a complete absence of all water molecules.

As used herein, “amorphous powder” refers to dry powders lacking a definite repeating form, shape, or structure, including all non-crystalline powders.

In one embodiment, the dry powder is a relatively cohesive powder which requires optimal deagglomeration condition. In one embodiment, the inhalation system provides a re-useable, miniature breath-powered inhaler in combination with single-use cartridges containing pre-metered doses of a dry powder formulation.

As used herein the term “a unit dose inhaler” refers to an inhaler that is adapted to receive or comprises a single container comprising a dry powder formulation and delivers a single dose of a dry powder formulation by inhalation from the container to a user. In some instances multiple unit doses will be required to provide a user with a specified dosage. In one embodiment, the inhaler is a dry powder inhaler, which can be disposable for single use, or reusable for multiple use with a single unit dose container.

As used herein the term “a multiple dose inhaler” refers to an inhaler having a plurality of containers, each container comprising a pre-metered dose of a dry powder medicament and the inhaler delivers a single dose of a medicament powder by inhalation at any one time.

As used herein a “container” is an enclosure configured to hold or contain a dry powder formulation, a powder containing enclosure, and can be a structure with or without a lid. This container can be provided separately from the inhaler or can be structurally integrated within the inhaler (e.g. non-removable). Further, the container can be filled with a dry powder. A cartridge can also include a container.

As used herein a “powder mass” is referred to an agglomeration of powder particles or agglomerate having irregular geometries such as width, diameter, and length.

As used herein, the term “microparticle” refers to a particle with a diameter of about 0.5 to about 1000 μm, irrespective of the precise exterior or interior structure. However four pulmonary delivery microparticles that are less than 10 μm are generally desired, especially those with mean particles sizes of less than about 5.8 μm in diameter.

In one embodiment, a pharmaceutical composition is provided comprising vasoactive intestinal peptide and a diketopiperazine, including 3,6-bis (4-fumaryl-4-aminobutyl)-2,5-diketopiperazine, or a salt thereof.

In an alternate embodiment, a method is provided for treating disease of the lungs, including, interstitial lung disease, for example, idiopathic pulmonary fibrosis, inflammation, acute or chronic respiratory distress syndrome, comprising, administering to a subject in need of treatment an inhalable composition comprising a vasoactive intestinal peptide, and a diketopiperazine of the formula:

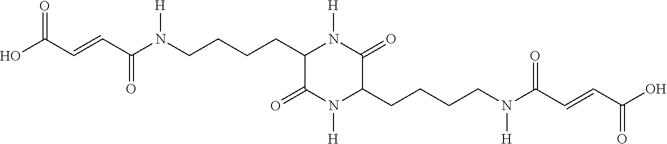 [SMILES: O=C(O)/C=C/C(=O)NCCCCC1NC(=O)C(CCCCNC(=O)/C=C/C(=O)O)NC1=O]

and optionally, one or more pharmaceutical excipients and/or carriers as define above with respect to the formulation. In one embodiment herewith, the formulation comprises vasoactive intestinal peptide, and a diketopiperazine such as 3,6-bis (4-fumaryl-4-aminobutyl)-2,5-diketopiperazine, or a salt thereof and optionally, one or more amino acid, wherein the amino acid is isoleucine, leucine, trileucine, cystine, cysteine, glycine, lysine, arginine, histidine, or methionine; and one or more sugars, including, lactose, mannitol, mannose, sorbitol, trehalose, and the like. In this and other embodiments, the diketopiperazine is in a dried crystal carrier form prior to making a suspension in water for use in the formulation, in other embodiments, others carriers can be used, including, saccharides, an oligosaccharides, or a polysaccharides, including lactose, trehalose, mannose, mannitol, or sorbitol; zinc citrate and ascorbic acid; wherein the formulation is made in solution and by a spray-drying process, wherein the peptide can be in a buffered solution having a pH ranging from about pH 3.5 to about pH 7; or pH 4.5 to pH 6.5.

In a further embodiment, the formulation is an amorphous dry powder comprising, microparticles of disodium fumaryl diketopiperazine comprising vasoactive intestinal peptide, a surfactant and an amino acid as disclosed above. In an embodiment, the formulation comprises an crystalline dry powder comprising a peptide, including, a heat-sensitive peptide, including vasoactive intestinal peptide; wherein the dry powder is formed by mixing vasoactive intestinal peptide in a solution containing a diketopiperazine suspension of crystalline particles at an adjusted pH ranging from pH 3.5 to about pH 5.0 and spray-drying the suspension.

In an exemplary embodiment, a dry powder formulation is provided, comprising, a peptide or a protein, wherein the peptide or protein is sensitive to degradation by oxidation or heat. In a particular embodiment, the dry powder formulation comprises a peptide including, vasoactive intestinal peptide, a derivative thereof, or an analog thereof; one or more than one amino acid, including, leucine, isoleucine and methionine; one or more than one sugar, including, mannitol, xylitol and trehalose and, optionally, one or more than one antioxidant, including, ascorbic acid, selenium, a carotenoid and vitamin E. In one embodiment herewith, the dry powder formulation comprises vasoactive intestinal peptide, a sugar, at least one amino acid and, optionally, an antioxidant, wherein the amino acid is methionine and/or leucine, the antioxidant is ascorbic acid and the sugar is mannitol.

In an alternate embodiment, the one or more pharmaceutically acceptable carriers are selected from sugars, for example, saccharides, disaccharides; oligosaccharides; an amino acid; wherein the sugar is, for example, trehalose, mannose, mannitol or sorbitol; polyethylene glycol, polyvinylpyrrolidone, and a diketopiperazine capable of forming microparticles, including, fumaryl diketopiperazine, succinyl diketopiperazine, maleyl diketopiperazine, malonyl diketopiperazine and oxalyl diketopiperazine, or the disodium, or magnesium salt thereof, and derivatives thereof.

In one embodiment, a stable formulation is provided for used as solutions or suspensions for nebulization, or as dry powders for inhalation. In an embodiment, the formulation comprises one or more unstable peptides for the treatment of lung inflammation, including, vasoactive intestinal peptide and/or a prostaglandin, PG 12, including a derivative or analog such as treprostinil, one or more amino acid, wherein the amino acid is isoleucine, leucine, trileucine, cystine, cysteine, glycine, lysine, arginine, histidine, or methionine; and one or more sugars, including, lactose, mannitol, mannose, sorbitol, trehalose, and the like. In this and other embodiments, the carrier can be a saccharide, an oligosaccharide, or a polysaccharides, including, lactose, trehalose, mannose, mannitol, or sorbitol; zinc citrate and ascorbic acid; wherein the formulation is made in solution and by a spray-drying process, wherein the peptide can be in a buffered solution having a pH ranging from about pH 3.5 to about pH 7; or pH 4.5 to pH 6.5. In this embodiment, the amount of vasoactive intestinal peptide is up to about 300 μg and the amount of treprostinil is up to about 200 μg in the composition per dosage form.

In this embodiment, the content of peptide, or protein, including, vasoactive intestinal peptide, for example, can be provided in the formulation in amounts ranging from about 0.1% (w/w) to about 50% (w/w), from about 0.5% (w/w) to about 40% (w/w); from about 0.5% (w/w) to about 20% (w/w); or from about 1% (w/w) to about 10% (w/w).

In one embodiment, there is provided a method for the effective delivery of a formulation to the lungs of a subject, comprising providing to a subject in need of treatment an inhalation system comprising, an inhaler, including, a cartridge containing a formulation comprising, a dry powder comprising up to 200 μg, or 10 wt % of vasoactive intestinal peptide; up to about 90 wt % mannitol; up to about 15 wt % leucine and up to about 5 wt % ascorbic acid. In this and other embodiments, the inhalation system delivers a powder plume comprising particles having a volumetric median geometric diameter (VMGD) less than 8 μm. In an example embodiment, the VMGD of the microparticles can range from about 4 μm to 6 μm. In an example embodiment, the VMGD of the powder particles can be from 3 μm to about 6 μm in a single inhalation of the formulation of fill mass ranging between 1 mg and 10 mg of dry powder. In this and other embodiments, the inhalation system delivers greater than 40%; or greater than 60% of the dry powder formulation from the cartridge. In particular embodiments herewith, the dry powder formulation can comprise from 10 μg to about 20 μg, from about 20 μg to about 50 μg, from about 50 μg to about 100 μg, from about 100 μg to about 150 μg, or from about 150 μg to about 200 μg of the active agent, including, VIP in the composition. In some embodiments, the dry powder formulation can comprise more than 200 μg depending on the patient's need.

Further embodiments concern drug delivery systems comprising an inhaler, a unit dose dry powder medicament container, and a dry powder comprising a heat-sensitive peptide as disclosed herein, including, a formulation comprising vasoactive intestinal peptide, methionine and ascorbic acid. In an embodiment, the formulation herein comprises vasoactive intestinal peptide, leucine, methionine and ascorbic acid.

One embodiment discloses a formulation comprising vasoactive intestinal peptide, a derivative thereof, or an analog thereof, wherein the formulation further comprises diketopiperazine microparticles, including, microparticles of fumaryl diketopiperazine having a specific surface area of less than about 67 m2/g. Another embodiment includes diketopiperazine microparticles in which the specific surface area is from about 35 to about 67 m2/g, within a 95% confidence limit. Another embodiment includes diketopiperazine microparticles in which the specific surface area is from about 35 to about 62 m2/g. Another embodiment includes diketopiperazine microparticles in which the specific surface area is from about 40 to about 62 m2/g. In one embodiment, the diketopiperazine has a trans isomer content ranging from 45% to about 59%, or from about 52 to about 57%.

In alternative embodiments, the FDKP microparticles comprise an unstable drug or active agent in solution or suspension. In various alternate embodiments of the FDKP microparticles, the drug can be, for example, a peptide, including, glucagon-like peptide-1 (GLP-1), glucagon, exendin, parathyroid hormone, calcitonin, oxyntomodulin, derivatives and/or analogs thereof, and the like. In another embodiment using the FDKP microparticles, the peptide content can vary depending on downstream processing conditions. In a particular example, the FDKP microparticles or other carrier particles can be prepared to have drug/peptide content that can vary depending on the dose to be targeted or delivered. For example, wherein the drug is vasoactive intestinal peptide, the peptide content component can be from about 1 μg to about 300 μg, 10 μg to about 200 μg, 30 μg to 150 μg, or 50 μg to 100 μg per dose in the powder formulation. In certain embodiments, the microparticles in suspension comprising pharmaceutically acceptable carriers and/or excipients can be spray-dry to make the stable formulations.

A further embodiment pertains to a drug delivery system comprising a combination of an inhaler, a unit dose dry powder medicament container, for example, a cartridge or capsule, and comprising the dry powder formulations disclosed herein with the active agent. In one embodiment, the delivery system for use with the dry powders includes an inhalation system comprising a high resistance inhaler having air conduits which impart a high resistance to airflow through the conduits for deagglomerating and dispensing the powder. In one embodiment, the inhalation system has a resistance value of, for example, approximately 0.065 to about 0.200 (kPa)/liter per minute. In certain embodiments, the dry powders can be delivered effectively by inhalation with an inhalation system wherein the peak inhalation pressure differential can range from about 2 to about 20 kPa, which can produce resultant peak flow rates of about between 7 and 70 liters per minute. In embodiments, the inhalation system is configured to provide a single dose by discharging powder from the inhaler as a continuous flow, or as one or more pulses of powder delivered to a patient. In some embodiments disclosed herewith, the dry powder inhaler system comprises a predetermined mass flow balance within the inhaler. For example, a flow balance of approximately 10% to 70% of the total flow exiting the inhaler and into the patient is delivered by one or more dispensing ports, which airflow passes through the area containing the powder formulation, and wherein approximately 30% to 90% air flow is generated from other conduits of the inhaler. Moreover, bypass flow, or flow not entering and exiting the area of powder containment such as through a cartridge, can recombine with the flow exiting the powder dispensing port within the inhaler to dilute, accelerate and ultimately deagglomerate the fluidized powder prior to exiting the mouthpiece. In one embodiment, flow rates ranging from about 7 to 70 liters per minute result in greater than 75% of the container or the cartridge contents dispensed in fill masses between 1 mg and 50 mg; or 1 mg to 30 mg. In certain embodiments, an inhalation system as described above can emit a respirable fraction/fill of a powder dose at percentages greater than 40% in a single inhalation, greater than 50%, greater than 60%, or greater than 70%.

In particular embodiments, an inhalation system is provided comprising a dry powder inhaler, and a dry powder formulation. In some aspects of this embodiment of the inhalation system, the dry powder formulation is provided in a unit dose cartridge. Alternatively, the dry powder formulation can be preloaded in the inhaler. In this embodiment, the structural configuration of the inhalation system allows the deagglomeration mechanism of the inhaler to produce respirable fractions greater than 50%; that is, more than half of the powder contained in the inhaler (cartridge) is emitted as particles of less than 5.8 μm. The inhalers can discharge greater than 85% of a powder medicament contained within a container during dosing. In certain embodiments, the inhalers can discharge greater than 85% of a powder medicament contained in a single inhalation. In one embodiment, the inhalers can discharge greater that 90% of the cartridge contents or container contents in less than 3 seconds at pressure differentials between 2 and 5 kPa with fill masses ranging up to 30 mg.

Another embodiment disclosed herein includes, a method of making microparticles suitable for pulmonary administration as a dry powder formulation comprising, a carrier particle, including, diketopiperazine microparticles. In this and other embodiments, the dry powder formulation is obtained by spray-drying a solution containing an unstable peptide, wherein the one or more excipients is dissolved in an aqueous solution comprising the zinc salt and citrate and mixed, followed by adding the amount of the peptide with mixing to form a feed solution; atomizing the flow of solution into a drying nitrogen gas flow at an inlet temperature of about 120° C. to 150° C. and an outlet temperature of about 60° C. to 65° C., or 50° C. to 75° C.

In some embodiments, the method of making diketopiperazine microparticles having the specific surface area of less than about 67 m2/g, and/or an a trans isomer ratio of about 45% to 65%, which utilizes a diketopiperazine having the formula 2,5-diketo-3,6-bis (N-X-4-aminobutyl) piperazine disodium salt or magnesium salt, wherein X is selected from the group consisting of fumaryl, succinyl, maleyl, and glutaryl. In an exemplary embodiment, the diketopiperazine has the formula (bis-3,6-(N-fumaryl-4 aminobutyl)-2,5-diketopiperazine or 2,5-diketo-3,6-bis (N-fumaryl-4-amino-butyl) piperazine.

Another embodiment disclosed herein includes a method of delivering a drug, for example, a peptide such as vasoactive intestinal peptide to a patient in need thereof comprising administering a dry powder to the deep lung by inhalation using a dry powder inhaler with the patient inhaling the dry powder; wherein the dry powder comprises diketopiperazine microparticles comprising vasoactive intestinal peptide, methionine and a citrate buffer; wherein the microparticles are formed of a diketopiperazine and have a surface area ranging from about 35 to about 67 m2/g or about 40 to about 67 m2/g and/or in microparticles having a trans isomer content raging from about 45% to about 65%.

Further embodiments involve methods of treating a pulmonary edema comprising administering to a patient in need of treatment a dry powder comprising having the patient inhale the dry powder from a breath powered inhaler containing the dry powder so that the dry powder in aerosol form reaches the lung. In some embodiments, the dry powder is delivered in one or more inhalations per treatment session. In particular embodiments, the treatment comprises delivering a dry powder in a single inhalation per dose.

In embodiments wherein a diketopiperazine is used as the carrier or excipient, fumaryl diketopiperazine 3,6-bis (N-fumaryl-4-aminobutyl)-2,5-diketo-diketopiperazine; FDKP) is preferred diketopiperazine for pulmonary applications, which has the formula:

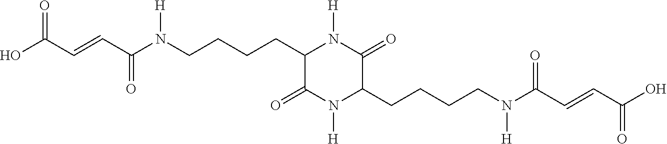 [SMILES: O=C(O)/C=C/C(=O)NCCCCC1NC(=O)C(CCCCNC(=O)/C=C/C(=O)O)NC1=O]

(E)-3,6-bis [4-(N-carbonyl-2-propenyl) amidobutyl]-2,5-diketopiperazine, or a pharmaceutically acceptable salt thereof, including, a disodium salt, a magnesium salt, a lithium and a potassium salt.

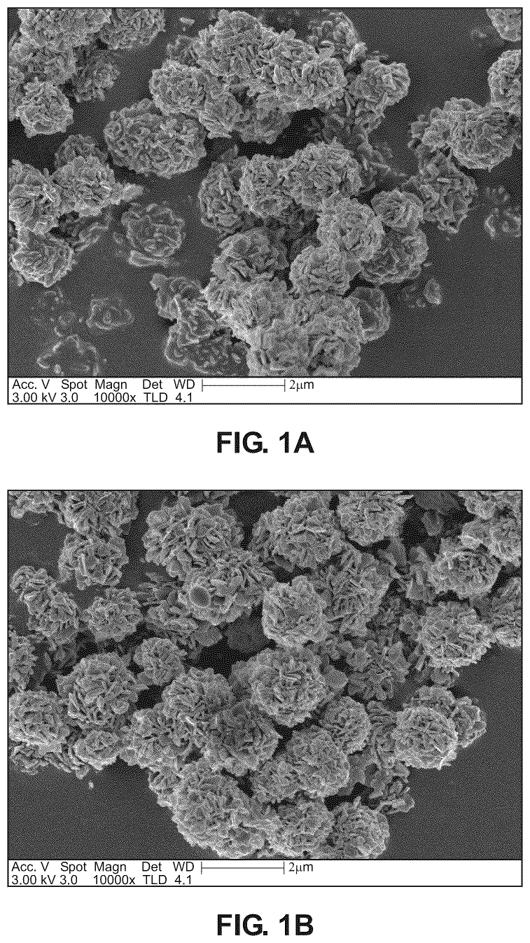

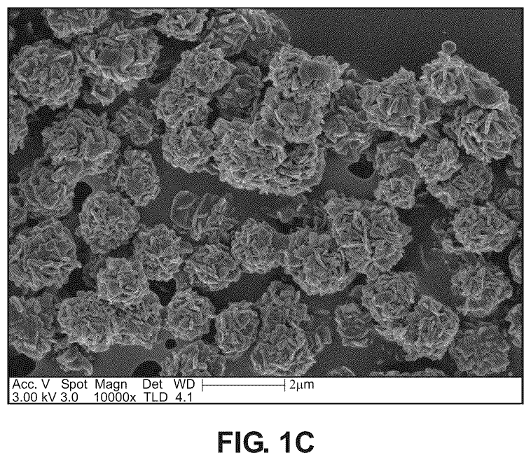

FDKP microparticles having a diameter of between about 0.5 and about 10 microns can reach the lungs, successfully passing most of the natural barriers. Particles in this size range can be readily prepared from diketopiperazines with acidic groups, such as the carboxylate groups in FDKP (as well as in related molecules such as 2,5-diketo-3,6-di (4-X-aminobutyl) piperazine wherein X is succinyl, glutaryl, or maleyl). Upon acid precipitation self-assembled particles composed of aggregates of crystalline plates are obtained. The size of these plates relates to the specific surface area of the particles which in turn is implicated in effects on the structure, loading capacity, and aerodynamic performance of the particles. FIG. 1 depicts scanning electron micrographs of dry powder particles comprising crystalline plates of (E)-3,6-bis [4-(N-carbonyl-2-propenyl) amidobutyl]-2,5-diketopiperazine and vasoactive intestinal peptide. As seen in the micrographs, the dry powder particles appear substantially homogeneous.

Embodiments disclosed herein show that microparticles having a specific surface area less than about 67 m2/g exhibit characteristics beneficial to delivery of drugs to the lungs such as improved aerodynamic performance. An alternate embodiment with a specific surface area less than about 62 m2/g provides a greater level of assurance that a batch of particles will meet a minimum aerodynamic performance standard. As SSA also affects drug loading/content capacity, various embodiments require SSA≥35, 40, or 45 m2/g for improved drug adsorption capacity. In some embodiments the SSA can less than 35 m2/g depending on the active agent used.

The microparticles described herein can comprise one or more active agents. As used herein “active agent”, used interchangeably with “drug”, refers to pharmaceutical substances, including small molecule pharmaceuticals, biologicals and bioactive agents. Active agents can be naturally occurring, recombinant, or of synthetic origin, including proteins, polypeptides, peptides, nucleic acids, organic macromolecules, synthetic organic compounds, polysaccharides and other sugars, fatty acids, and lipids. The active agents that are unstable can fall under a variety of biological activity and classes, that can be used for treating an array of diseases or disorders, including, vasoactive agents, neuroactive agents, hormones, anticoagulants, immunomodulating agents, cytotoxic agents, antibiotics, antiviral agents, antigens, infectious agents, inflammatory mediators, hormones, and cell surface antigens. In a particular embodiment herewith, the compositions are exemplified with vasoactive intestinal peptide for treating inflammatory disease in the lungs, including pulmonary edema.

The drug content to be delivered depends on the need of the subject and the potency of the drug. In certain embodiments, microparticles formed from FDKP having a trans isomer content between 45% and 65% is typically greater than 0.01% are used. In one embodiment, the drug content to be delivered with the microparticles having the aforementioned trans isomer content, can range from about 0.01% to about 50%, which is typical for peptides such as vasoactive intestinal peptide.

The range of drug content to be delivered depends on the disease to be treated and the severity of the disease in the patient. For example, for pulmonary delivery, the drug content in the formulation to be used is typically between about 0.01% and about 90%, depending on the form and size of the drug to be delivered and the potency of the dose required. For vasoactive intestinal peptide, preferred loads are about 0.5 wt % to about 50 wt %, or from about 1 wt % to about 20 wt %. In some embodiments, the formulations can be delivered with alternative inhaler embodiments.

### EXAMPLES

The following examples are included to illustrate embodiments of the disclosed formulation and methods. It should be appreciated by those of skill in the art that the techniques disclosed in the examples, which follow represent techniques discovered by the inventor to function well in the practice of the present disclosure, and thus can be considered to constitute preferred modes for its practice. However, those of ordinary skill in the art should, in light of the present disclosure, appreciate that many changes can be made in the specific embodiments which are disclosed and still obtain a like or similar result.

### Example 1

Preparation, characterization and stability of vasoactive intestinal peptide dry powders: Vasoactive Intestinal Peptide (VIP) is a peptide consisting of 28 amino acid residues, which was purchased from American Peptide Company, Inc. of Sunnyvale, CA. Crystalline particles of fumaryl diketopiperazine (FDKP) were prepared and suspended in water and mixed with a VIP solution, then flash frozen as pellets in liquid nitrogen and lyophilized to produce sample powders. Three powders prepared with target peptide loads or content of 5%, 10%, and 20% and when assayed, resulting in powders having VIP content of 5.6%, 10.7% and 20.8% (Table 1 below). The RF/fill (cartridge emptying) were 56% (88%), 47% (76%), and 22% (95%), for respective target loads of 5, 10, and 20%, indicating that can be aerosolized and form particles, which are suitable for lung delivery. Samples of the powders were as prepared were also used for scanning electron microscope (SEM) analysis (RJ Lee Group, Inc.). Results of these experiments are described below and with respect to FIG. 1. FIGS. 1A, 1B and 1C show the physical characteristics of the crystalline FDKP powder morphology containing various amounts of VIP, respectively, 5.6 wt %, 10.7 wt %, and 20.8 wt %. The SEMs show that the particles appear to remain the same at the different amounts of the VIP peptide and there appears to be no discernable change in their morphology.

VIP Powder Preparation: All powders subsequently herewith made were produced in the same manner to a target yield of 1000 mg. For example, for the 10% target content, an FDKP particle suspension (9012 mg 10.00% solids=901.2 mg FDKP crystalline particles) was weighed into a 20 mL clear glass vial. The suspension was capped and mixed using a magnetic stirrer to prevent settling of the precipitate. VIP solution (1001 mg of 10% peptide in 3 wt % acetic acid) was added to the vial and allowed to mix. The final composition ratio was approximately 10:90 VIP: FDKP particles. The VIP-FDKP suspension was adjusted to pH 4.49 by adding aliquots of 15% aqueous ammonia solution. The suspension was pelleted into a small crystallization dish containing liquid nitrogen. The dish was placed in a freeze dryer and lyophilized. The shelf temperature was ramped from −45° C. to 25° C. and then held at 25° C. for approximately 10 hours. The material was transferred to a clear glass vial. Total yield of the powder after transfer to the vial was 993 mg. Actual yield for all powders was between 985 and 1000 mg (98.5-100% yield). Samples of the various powders were taken and analyzed by various methods.

Powder Analysis: Determination of percent peptide content was performed by high pressure liquid chromatography (HPLC) using a Phenomenex Gemini C18 column and a mobile phase A of water containing 0.1% trifluoroacetic acid (TFA) and acetonitrile containing 0.1% (TFA); and the resultant data was analyzed using Waters Empower™ software.

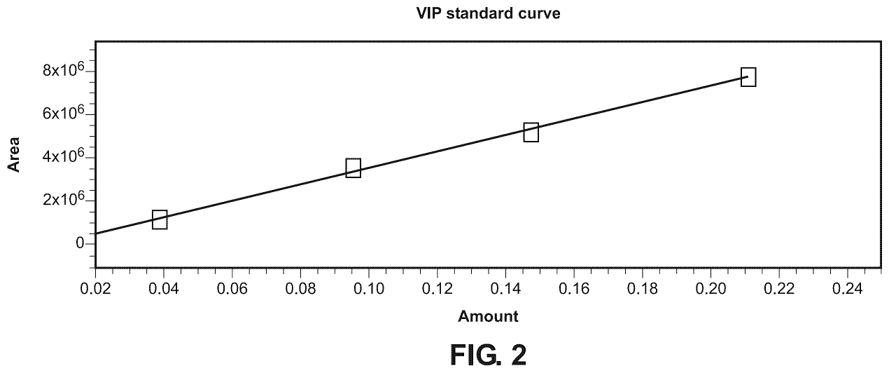

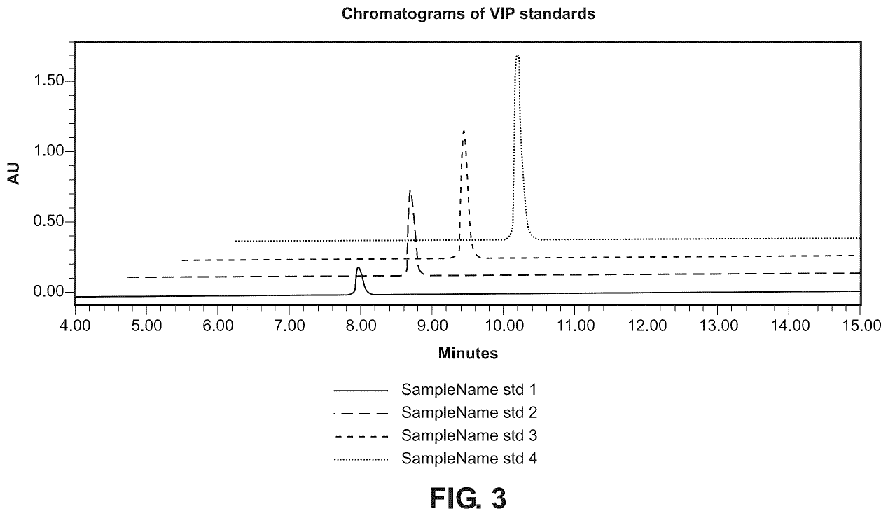

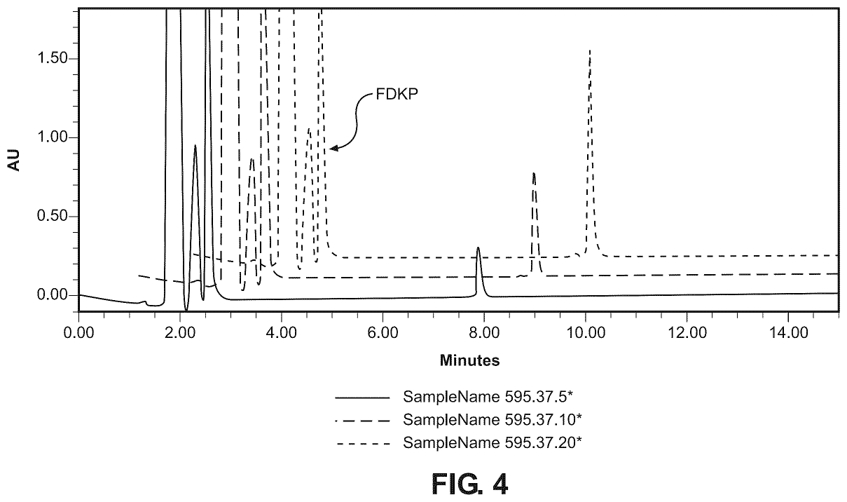

Sample powders were prepared at 1 mg/mL in 200 mM sodium phosphate, pH 7.5. Four VIP standards were prepared independently in 200 mM sodium phosphate, pH 7.5 from VIP API. The standard curve is shown in FIG. 2, which shows the content of VIP in the formulation is linear without the FDKP particles reaching saturation at the amount tested. FIG. 3 depicts data from the HPLC chromatograms of the VIP standards showing a single peak per standard tested. FIG. 4 demonstrates the sample dry powders made containing various crystalline FDKP-VIP target contents, i.e., 5% (1=______-), 10% (2=_ _ _) and 20% (3= - - - - ) and their corresponding HPLC chromatograms. Table 1 depicts some of the characteristics of the powders made and the performance of the powders upon testing using a dry powder inhaler.

TABLE 1
Properties of prototype FDKP-|VIP sample powders

| | Target Load | Calculated | % RF/Fill | % cartridge |
|---|---|---|---|---|
| Sample ID | (%) | Load (%) | contents | emptying |
| 1 | 5 | 5.6 | 56 | 88 |
| 2 | 10 | 10.7 | 47 | 76 |
| 3 | 20 | 20.8 | 22 | 95 |

The data show the powders delivered from 76% to 95% of the powder content from the dry powder inhalers used from 5.6% to 20% VIP content, which indicate that the powder were aerosolizable. The percent RF/Fill indicates the percentage of the particles that can reach the lungs due to their size. The data demontrate that the 5.6% sample comprises the highest percentage of particles that can reach the lungs at 56%. The data also demonstrate that the dry powders could be loaded accurately and that aerodynamic performance decreases with increasing amount of peptide content.

### Example 2

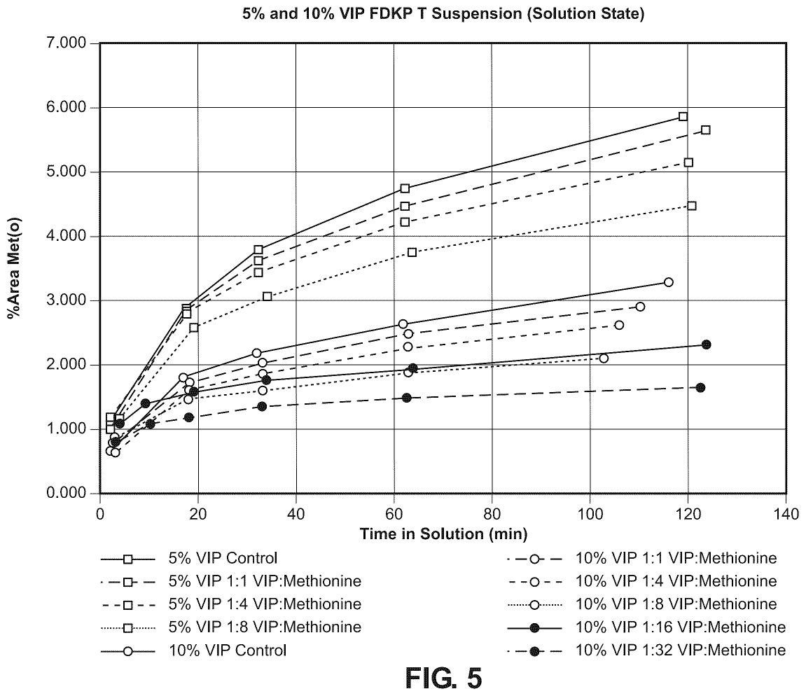

VIP FDKP-Suspensions Containing Methionine: Methionine (methionine was added at levels from 0.0 g to 0.0358 g) (methionine was added so the mole ratio of vasoactive intestinal peptide (VIP): methionine ranged from 1:0, 1:1, 1:4 1:8, 1:16 and 1:32) was dissolved in 3% aqueous acetic acid solutions (acetic acid solution was added at levels from 0.54 g to 1.08 g depending on the VIP charge used to prepare a 5% or 10% VIP solution in the acetic acid solution). A 10% solution of VIP was prepared by adding VIP (VIP was added at levels ranging from 0.05 g to 0.25 g) to the methionine in 3% aqueous acetic acid solution. Crystalline (T) FDKP-suspension (amount of T-suspension used ranged from 9.8 g to 23.46 g and varied based on the methionine and VIP amounts added to the suspension) (T-suspension used contained 8.61% solids) was added to the VIP/methionine/acetic acid solutions. These mixtures were stirred until uniform suspensions were obtained. Samples of the suspensions were assayed for stability for VIP content by reversed phase (RP) HPLC to monitor the levels of Met (O)-VIP formation. The remaining suspension was spray dried or lyophilized to form powders. Stability samples were analyzed, and the results are shown in FIG. 5 for 5 wt % and 10 wt % VIP with various concentrations of methionine compared to controls. FIG. 5 demonstrates that increasing methionine content in the solution increased the stability of the VIP peptide in the solution or suspension and thus it would also increase the stability of the dry powders.

### Example 3

Preparation of 10% VIP Methionine FDKP-Powders: Methionine (methionine charge was varied between 0.18 g and 0.36 g corresponding to a mole ratio of VIP: methionine of 1:16 and 1:32 respectively) was dissolved in 3% aqueous acetic acid solutions (4.05 g of 3% acetic acid solution was used to dissolve the methionine at the lower level and 6.75 g was used to dissolve the higher level of methionine). VIP (0.25 g) was added to the methionine/acetic acid solutions and was stirred until it completely dissolved. T-suspension (8.61% solids content) (T-suspension charge was 23.46 g for the formulation containing the lower amount of methionine and 21.37 g for the formulation containing the higher methionine charge) was added to the VIP/methionine/acetic acid solutions. The resulting suspensions were stirred until uniform and then were pelletized into liquid nitrogen. The pelletized suspensions were dried by lyophilization using a program, which, under full vacuum, started at −40° C. and increased the temperature by 0.2° C./min. to 25° C. where the temperature was held until each powder was completely dried.

### Example 4

Preparation of VIP Powders comprising Mannitol: Several solutions containing VIP were prepared simulating a 10% VIP dry powder formulation and periodically analyzed by reversed phase (RP) HPLC to evaluate the chemical stability of Patent Application 1951300.00635 PCT Patent Application Docket No. MKC-127 (P) (PCT) these solutions. Solutions containing mannitol alone, leucine alone, and in combination were evaluated. Two solutions containing two different antioxidants, methionine and ascorbic acid were also evaluated.

Solution Preparation: In the experiments, all the solutions were prepared targeting a 10% VIP powder formulation as shown in Table 2 below. For example, a 10% mannitol solution was prepared by adding 1 g of mannitol to 9 g of water, and a 1% leucine solution was prepared by adding 0.1 g of leucine to 9.9 g of water. The XC suspension contained 1.97% solids. For each solution, 5 mg of VIP was weighed into a small vial and the other reagents were added following the Table 2 below. Solutions were stirred continuously at room temperature. At each time point, a small aliquot was taken and diluted into a diluent (0.2 M sodium phosphate, 3 mM methionine, pH 7), and analyzed by RP-HPLC. An FDKP-VIP crystalline composite (XC) particle suspension was also prepared as detailed above and analyzed as described herewith.

TABLE 2

| | | Amount | % w/w Dry |
|---|---|---|---|
| Sample | Reagent | Added (mg) | Weight Basis |
| A | VIP | 5 | 10.0 |
| | Mannitol | 450 | 90.0 |
| | Total | 455 | |
| B | VIP | 5 | 10.0 |
| | Leucine | 4500 | 90.0 |
| | Total | 4505 | |
| C | VIP | 5 | 10.0 |
| | XC | 2300 | 90.0 |
| | Total | 2305 | |
| D | VIP | 5 | 10.0 |
| | Mannitol | 400 | 80.0 |
| | Leucine | 500 | 10.0 |
| | Total | 905 | |
| E | VIP | 5 | 10.0 |
| | Mannitol | 375 | 75.0 |
| | Leucine | 500 | 10.0 |
| | Methionine | 2.5 | 5.0 |
| | Total | 882.5 | |
| F | VIP | 5 | 10.0 |
| | Mannitol | 395 | 79.0 |
| | Leucine | 500 | 10.0 |
| | Ascorbic acid | 0.5 | 1.0 |
| | Total | 900.5 | |

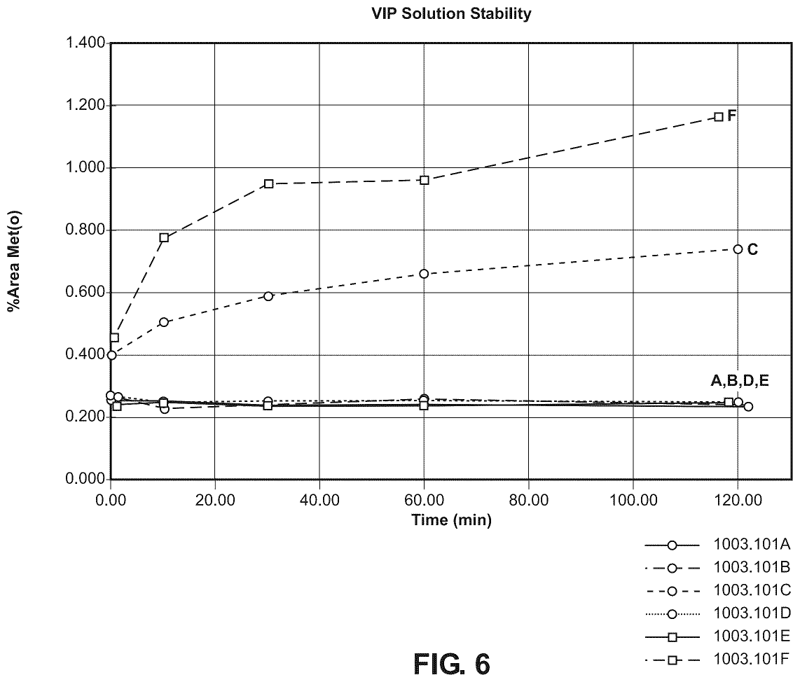

The solution stability experiments were carried out as described above and results with mannitol, leucine, methionine and XC suspension are shown in FIG. 6. As illustrated in FIG. 6, samples containing mannitol (Sample A) leucine (Sample B), and mannitol plus leucine sample (Sample D) showed almost no degradation over the course of 2 hours. Samples containing the antioxidant methionine (Sample E) also showed almost no degradation over the course of 2 hours. Sample C, the XC suspension had less degradation than the suspension containing the addition of ascorbic acid (Sample F). Surprisingly, the results of the ascorbic acid (Sample F) showed more degradation of the VIP as it is an oxygen scavenger. Perhaps, this was due to the concentration used in the example.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects those of ordinary skill in the art to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above cited references and printed publications are herein individually incorporated by reference in their entirety.

Further, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described

What is claimed:

1. A dry powder for treating lung disease comprising, an inhalable dry powder pharmaceutical formulation comprising vasoactive intestinal peptide, a derivative thereof, an analog thereof, or a salt thereof, one or more amino acids, one or more antioxidants, and/or a pharmaceutically acceptable carrier or excipient.

2. The dry powder of claim 1, wherein the vasoactive intestinal peptide is up to 300 μg for treatment session.

3. The dry powder of claim 1, wherein the pharmaceutically acceptable carrier is a sugar selected from the group consisting of mannose, mannitol, trehalose, and sorbitol.

4. The dry powder of claim 1, wherein the pharmaceutically acceptable carrier is a mannitol.

5. The dry powder of claim 1, wherein the pharmaceutically acceptable carrier is a diketopiperazine.

6. The dry powder of claim 4, wherein the diketopiperazine is fumaryl diketopiperazine, or succinyl diketopiperazine.

7. The dry powder of claim 1, wherein the one or more amino acids is selected from the group consisting of lysine, glycine, leucine, isoleucine, trileucine, histidine, and methionine.

8. The dry powder of claim 7, wherein the one or more amino acids are leucine and methionine.

9. The dry powder of claim 1, wherein the one or more amino acids or antioxidants is methionine.

10. The dry powder of claim 1, wherein the one or more antioxidants is selected from the group consisting of ascorbic acid, vitamin E, selenium, methionine and carotenoids.

11. The dry powder of claim 1, wherein a dose of the inhalable dry powder pharmaceutical formulation comprises up to 200 μg of vasoactive intestinal peptide, derivative thereof, analog thereof, or a salt thereof and up to 90 wt % mannitol.

12. An inhalable pharmaceutical formulation comprising mannitol, leucine, methionine and vasoactive intestinal peptide, an analog thereof, or a derivative thereof, in a dose of up to 200 μg for the treatment of edema in a subject.

13. The inhalable pharmaceutical formulation of claim 12, wherein the inhalable pharmaceutical formulation is manufacture in the dose is to be administered at least once a day.
